(19) United States
(12) Patent Application Publication
(10) Pub. No.: US 2024/0050339 A1
(43) Pub. Date: Feb. 15, 2024

(54) FORMULATION COMPRISING P-MENTHANE-3,8-DIOL

(71) Applicant: WORKFORCE BIOLOGICS LTD., Hertfordshire (GB)

(72) Inventor: John Woodruff, Poole (GB)

(21) Appl. No.: 18/269,010

(22) PCT Filed: Dec. 15, 2021
(86) PCT No.: PCT/EP2021/086010

§ 371 Date: Jun. 22, 2023

(30) Foreign Application Priority Data
Dec. 23, 2020 (GB) 2020461.6
Jun. 8, 2021 (GB) 2108165.8

(51) Int. Cl. A61K 8/34; A61K 8/06; A61K 8/37; A61K 8/60; A61Q 17/00

(52) U.S. Cl. CPC A61K 8/345; A61K 8/06; A61K 8/37; A61K 8/60; A61Q 17/005

(57) ABSTRACT

Described herein are compositions including p-Menthane-3,8-diol (PMD) having antiviral properties. The compositions are formulated to be stable and sprayable. Other formulations may be formulated as a topical barrier preparation.

### FIELD OF THE INVENTION

The present invention relates to an antiviral formulation; and more specifically to a stable, and in some embodiments sprayable formulation comprising p-Menthane-3,8-diol (PMD).

### BACKGROUND TO THE INVENTION

The insect-repellent effect of essential oils from the lemon eucalyptus tree (Corymbia citriodora, formerly known as Eucalyptus citriodora) has been known for some time. The active ingredient of these oils is PMD (p-Menthane-3,8-diol; also known as menthoglycol, or 2-(2-Hydroxypropan-2-yl)-5-methylcyclohexan-1-ol). The structure of PMD is shown below.

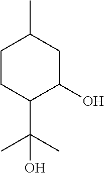

Note that PMD may exist in a number of different stereoisomeric forms.

The natural oils may be refined in order to increase the PMD content; the refined oils may be sold under a variety of trade names, including Citridiol™. It is believed that the natural oils may contain a mixture of different stereoisomers in varying ratios, depending on the source of the oil.

It is also possible to use synthetic PMD; for example, synthetic PMD may be obtained as described by Zimmerman and English in J. A. C. S. 75 (1953) pp 2367-2370. PMD is also a precursor obtained during the synthesis of menthol. The precursor is usually in the form of a specific isomer of PMD.

As well as having insect-repellent properties, PMD is also useful as an antiviral or viricidal agent. This is described, for example, in U.S. Pat. No. 7,872,051, which publication indicates that compositions comprising PMD are effective against influenza, Urbani Severe Acute Respiratory Syndrome (Urbani SARS) and HSV-1. The suggestion is also made that, since all these viruses have a lipid envelope, the compositions may also be effective against other viruses having a lipid envelope, including coronaviruses.

This suggestion is also supported by recent work by the UK's Defence Science and Technology Laboratory (Dstl), in which the effectiveness of an off-the-shelf insect repellent formulation containing PMD (Mosi-Guard™ Natural spray) or Citriodiol™ against the SARS-Cov2 virus—responsible for the COVID-19 pandemic—was assessed. The study reported that one minute liquid suspension tests indicated that Citriodiol™ ethanol, isopropanol and Mosi-guard™ Natural have anti-viral activity against SARS-CoV-2 England-2 isolate if mixed with the virus in the liquid phase; and that SARS-CoV-2 England 2 isolate survival studies on latex as a ‘synthetic skin’ (which was pre-treated with Mosi-guard™ Natural approximately 1 hour before testing) provided evidence of anti-viral properties against SARS-CoV-2 England-2 isolate. The work is reported at https://www.gov.uk/government/publications/experimental-survival-of-sars-cov-2-on-an-insect-repellent-treated-surface-3 (accessed 2 Dec. 2020), and also in Smither et al, Journal of General Virology, 2021, vol 102 issue 4; doi:10.1099/jgv.0.001585.

It would be useful to provide formulations comprising PMD suitable for use as antiviral agents. However, off-the-shelf products intended for use as insect repellents, or other Citriodiol™-containing products such as sunscreens, are generally inappropriate for repurposing as antiviral agents, for reasons including mode of application, consistency, stability, and so on. Furthermore, insect repellent formulations typically have relatively high levels of Citriodiol™—up to 30%—and may contain additional components. It is therefore an object of the present invention to provide PMD formulations for use as antiviral agents. Particularly preferred formulations are stable and sprayable, to enable easy transport, storage, and application over a wide area. In other embodiments, a stable formulation as described herein may be incorporated into a non-sprayable preparation such as a cream, gel, or lotion, for topical application to the skin.

### SUMMARY OF THE INVENTION

The present invention provides a number of embodiments of formulations comprising PMD which are believed to be suitable for use as antiviral agents. While preferred embodiments should be stable and sprayable (for example, as a mist or a fog) to allow wide coverage, in other embodiments the formulation may be incorporated into a non-sprayable preparation, for example for topical application to the skin. It was found when researching possible formulations that PMD showed a tendency to crystallise, in particular when used at concentrations lower than those which may be found in conventional insect repellents, such that potential formulations required to be modified in order to reduce crystallisation. The skilled person will appreciate that crystallised PMD may interfere with spray delivery mechanisms, so this may be a significant obstacle to practical widespread use as an antiviral; or of course may reduce the effectiveness of a preparation as an antiviral.

Certain antiviral compositions of the invention are intended to be sprayable. Thus, the compositions can be delivered from non-aerosol mechanical pump spray devices or from pressurized aerosol canisters using a propellant or from any other mechanism or method of dispersing compositions. The compositions may be intended for topical application to the skin, and/or for application to surfaces, and/or for misting to fill a volume. A wide range of mechanical pump spray devices and aerosol canister systems are well known to those of ordinary skill in the art. Similarly, a wide range of propellant materials are well known to those skilled in the art. Nonlimiting examples include lower molecular weight hydrocarbons such as propane n-butane and isobutane, nitrogen, carbon dioxide, nitrous oxide, and so forth, as well as compressed or pressurised air. Mixtures of such propellants can also be used.

Unless otherwise indicated, the concentrations of ingredients specified below are given in terms of the weight of the ingredient based on the weight of the sprayable antiviral composition as a whole but excluding any propellant that might be present.

According to a first aspect of the present invention, there is provided a sprayable antiviral composition, the composition comprising:
a) from 0.1% to 25% by weight of the total composition of p-Menthane-3,8-diol (PMD);
b) from 5% to 40% by weight of the total composition of one or more solvents selected from short chain (C1-05) primary alcohols and short chain (C3-05) secondary alcohols, and combinations thereof;
c) either i) from 0.5% to 10% by weight of the total composition of a glyceride-based emulsifier; or
   ii) from 10% to 30% by weight of the total composition of a non-ionic ethoxylated sorbitan ester surfactant.

It is to be understood that the term “antiviral”, as used herein, means “having the capacity to inhibit or stop the growth and reproduction of viruses”, or “having the capacity to destroy or inactivate viruses”. It is believed that PMD may have further useful properties beyond the antiviral activity, including potentially bacteriocidal, fungicidal and anti-inflammatory properties. It will therefore be understood that the formulations—both sprayable and topically-applied—described herein may also be useful for bacteriocidal, fungicidal, and/or anti-inflammatory purposes. Further, the combination of bacterial and/or fungal colonisation with chronic inflammation lie at the heart of the pathology of a number of clinical challenges we are faced with in hospitals and the community. For example burn wounds, eczema, psoriasis, intertrigo and most commonly of all, teenage acne. We therefore believe that the compositions of the invention are suited to the treatment of such conditions, and the invention further provides a composition as herein described for use in the treatment of a condition characterised by bacterial and/or fungal colonisation with chronic inflammation; and in particular for use in the treatment of a condition selected from burn wounds, eczema, psoriasis, intertrigo and acne.

The composition may further optionally comprise:
d) from 2% to 10% by weight of a polar ester; and/or
e) from 0.1% to 3% of an emulsion stabiliser (non-limiting examples include carbomers, cellulose gun, xanthan gum and microcrystalline cellulose)

The balance of the composition may be water, and optional ingredients to improve shelf life and aesthetic properties.

In preferred embodiments, the composition consists essentially of components a) to c), and optionally d) and/or e), and water. That is, no additional components are present. In particular this means the composition can be provided as an oil-free, and (in some embodiments) ethanol-free composition. In other embodiments the composition may consist essentially of components a) to c), and optionally d) and/or e), and water, and further optional ingredients to improve shelf life and aesthetic properties.

The composition may be in the form of an emulsion, when an emulsifier is used, or in the form of a solution, where no emulsifier is used. Preferred embodiments provide the composition as a solution, since this is easier to handle and manipulate, but there may be some circumstances when an emulsion is preferred. In particular, an emulsion may be preferable when the composition is to be formulated on site (for example, to be prepared by the end user or an intermediary), rather than being centrally manufactured and distributed.

The PMD present in the composition may be any single isomer or any combination of one or more isomers. In preferred embodiments, the PMD is at least 90%, 92%, 93%, 94%, 95%, or more of a pure single isomer. However, we believe that the relevant antiviral properties are not dependent on a particular isomer, such that other degrees of purity or mixtures of isomers may be used.

The amount of PMD present in the composition may be from 0.1%-25%, preferably 0.5%-10%, alternatively 1%-9%, preferably 2%-8%, or 3%-7%, or 4%-6%. In preferred embodiments, the composition comprises 5% by weight of PMD.

The solvent may be selected from short chain (C1-05) primary and short chain (C3-05) secondary alcohols, and mixtures thereof. The term alcohols includes diols and polyols. Possible primary alcohols include ethanol or methanol; however, given the potential for abuse of formulations containing these, their use is not preferred. Preferred secondary alcohols are propanols, and particularly preferred are propan-2-ol (also referred to as isopropyl alcohol) and propane-1,2-diol (also referred to as propylene glycol); and indeed these are each particularly preferred in embodiments of the invention. Other alcohols which may be used include (in a non-limiting list) ethylene glycol, dipropylene glycol, butylene glycol, and capryl glycol.

The solvent may be present from 5% to 40% by weight of the total weight of the composition. Where a single solvent is present—for example, propan-2-ol or propane-1,2-diol—the preferred amount of solvent present is from 5-20% by weight, more preferably 10-20%, yet more preferably 13-18%, and most preferably 15% by weight. Either of these solvents may be used individually in this amount. Where a mix of solvents is used, then these may be used in any desired ratio, and a preferred amount of solvent is from 20-40% by weight, more preferably 25-35% by weight, and most preferably 30% by weight. One example of a preferred composition includes 25% propan-2-ol and 5% propane-1,2-diol; this is more preferred where the composition is not an emulsion. Hence in a further embodiment, when the solvent is or includes propane-1,2-diol, component c) is not an emulsifier. We have found that propan-2-ol alone is an efficient solvent, but that addition of propane-1,2-diol can help to reduce loss by evaporation and the propensity for the PMD to crystallise and potentially block dispensing of the composition.

The glyceride-based emulsifier, when present, may be present at 0.5-10% by weight; preferably 0.5-5%, more preferably 1.5%-3.5%, and still more preferably 2.5%. Preferred emulsifiers include polyglyceryl-ester emulsifiers or glyceryl esters of fatty acids; one particularly preferred emulsifier is glyceryl oleate citrate, with alternatives being glyceryl stearate citrate or glyceryl cocoate citrate. Commercially-available emulsifiers may be used; for example, that available under the brand “dermofeel® easymuls plus” from Evonik (which contains glyceryl oleate citrate). Other emulsifiers which may be suitable include: the “Sucrabase” range produced by Alfa Chemicals (comprising caprylic/capric triglycerides, glycerine, sucrose laurate, sucrose stearate); DUB Base Expert+ from Stearinerie Dubois (containing glyceryl stearate citrate, sucrose stearate, polyglyceryl-4 cocoate, cetyl alcohol, sodium ricinoleate). Where commercial formulations of emulsifiers are used, it is preferred that these are PEG free.

The non-ionic ethoxylated sorbitan ester surfactant, when present, may be present at 10-30% by weight, preferably 10-20%, more preferably 12.5-17.5%, and most preferably around 15%. Preferred surfactants are polysorbates, in particular polysorbate 80, but in certain embodiments other polysorbates may be used such as polysorbate 20, polysorbate 40, polysorbate 60. Polysorbates are a class of surfactant comprising ethoxylated esters. Polysorbate 80 is a mixture of oleate esters of sorbitol and sorbitol anhydrides, consisting predominantly of the monoester, condensed with approximately 20 moles of ethylene oxide. It is also known as Polyoxyethylene (20) Sorbitan Monooleate. The higher the degree of ethoxylation the more likely it will be a good solubiliser.

In certain embodiments, a polar ester may be present, from 2% to 10% by weight; preferably from 3-9%, from 4-8%, or most preferably 5%. We have found that addition of such an ester may help reduce crystallisation of the PMD. A preferred polar ester is C12-15 alkyl benzoate, but others may be selected; for example, from those commonly used in cosmetics. Examples of other esters which may be useful in the invention include Butyl Stearate, Butyloctyl Salicylate, C12-13 Alkyl Lactate, Cetearyl Ethylhexanoate, Cetearyl Isononanoate, Cetearyl Nonanoate, Cetyl Dimethyloctanoate, Cetyl Ethylhexanoate, Cetyl Isononanoate, Cetyl Palmitate, Decyl Isostearate, Decyl Oleate, Diethyl hexyl Adipate, Diethyl hexyl Carbonate, Diisopropyl Adipate, Diisostearyl Malate, Ethyl Oleate, Ethylhexyl Palmitate, Isocetyl Ethylhexanoate, Isocetyl Isodecanoate, Isocetyl Stearoyl Stearate, Isodecyl Ethylhexanoate, Isodecyl Isononanoate, Isodecyl Neopentanoate, Isononyl Isononanoate, Isopropyl Acetate, Isopropyl Arachidate, Isopropyl Behenate, Isopropyl Benzoate, Isopropyl Citrate, Isopropyl Cyanoacrylate, Isopropyl Cyclohexylpropionate, Isopropyl Hydroxystearate, Isopropyl Isostearate, Isopropyl Laurate, Isopropyl Linoleate, Isopropyl Maleate, Isopropyl Myristate, Isopropyl Nicotinate, Isopropyl Oleate, Isopropyl Palmitate, Isopropyl Pelargonate, Isopropyl Ricinoleate, Isopropyl Sorbate, Isopropyl Stearate, Isopropylacrylamide, lsopropylbenzyl Salicylate, Isosteareth-3 Ethylhexanoate, Isostearyl Ethylhexanoate, Isostearyl Isononanoate, Isostearyl Isostearate, Isostearyl Neopentanoate, Isotridecyl Isononanoate, Myristyl Lactate, Myristyl Myristate, Octyldodecyl Lactate, Octyldodecyl Stearate, Phenethyl Benzoate, PPG 3 Benzyl Ether Myristate, Propylene Glycol Dibenzoate, Stearyl Caprylate, Stearyl Heptanoate, Triethylhexanoin, Triisononanoin, Triisopropyl Citrate, and Triisopropyl Trilinoleate. Particularly preferred examples may include Decyl Oleate, Diethyl hexyl Carbonate, Isopropyl Benzoate, Isopropyl Citrate, Isopropyl Myristate, and Isopropyl Palmitate.

Particularly preferred embodiments of the invention are, first, a sprayable antiviral composition, the composition comprising:
a) from 0.5% to 10%, preferably 3-7%, more preferably 5%, by weight of the total composition of p-Menthane-3,8-diol (PMD);
b) from 10% to 30%, preferably 15-20%, more preferably 20%, by weight of the total composition of propan-2-ol;
c) from 10% to 30%, preferably 10%, by weight of the total composition of polysorbate 80;
d) the balance is water.

A second particularly preferred embodiment is a sprayable antiviral composition, the composition comprising:
a) from 0.5% to 10%, preferably 3-7%, more preferably 5%, by weight of the total composition of p-Menthane-3,8-diol (PMD);
b) 20% by weight of the total composition of propan-2-ol;
c) from 10% to 30%, preferably 10%, by weight of the total composition of polysorbate 80;
d) 10% by weight of the total composition of propane-1,2-diol;
e) the balance is water.

An embodiment of the invention provides a sprayable antiviral composition, the composition comprising:
a) 5% by weight of the total composition of p-Menthane-3,8-diol (PMD);
b) 15-30% by weight of the total composition of propan-2-ol;
c) from 10% to 20% by weight of the total composition of polysorbate 80;
d) 0-10% by weight of the total composition of propane-1,2-diol; wherein, when propane-1,2-diol is present, the total combined amount of propane-1,2-diol and propan-2-01 is 30%;
e) the balance is water.

An embodiment of the invention provides a sprayable antiviral composition, the composition comprising:
a) 5% by weight of the total composition of p-Menthane-3,8-diol (PMD);
b) 10-15% by weight of the total composition of propane-1,2-diol;
c) 0-5% by weight of the total composition of a polar ester;
d) 1-5% by weight of the total composition of an emulsifier;
e) the balance is water.

A further aspect of the invention provides a concentrate which, when diluted with water, yields a sprayable antiviral composition as defined herein. The concentrate cannot be water-free but will preferably contain a minimum amount of water required to provide a stable solution or paste. This can then be diluted as required to yield the composition. In certain embodiments, we believe that a maximum of 25% by weight of water in the concentrate is preferred.

A still further aspect of the invention provides a spray bottle containing a composition as defined herein.

Further aspects of the invention relate to the incorporation of compositions as described into a (preferably non-sprayable) formulation for topical application to the skin; for example, a gel, cream, or lotion. Incorporation of the composition into a gel, cream, or lotion for topical application to the skin allows for a longer-term barrier preparation to be used, which we believe in turn will permit longer lasting antiviral effects. The stable antiviral compositions as described may be added to a suitable base for a gel, cream, or lotion; such bases will be known to those of skill in the art. For example, a simple face cream base may include water, emulsifying wax, and oil, in appropriate proportions to achieve a desired thickness of the cream. Examples of emulsifying waxes may include Emulsimulse (Glyceryl Stearate, Cetearyl Alcohol, and Sodium Stearoyl Lactylate); Emulsifying Wax NF (Cetostearyl Alcohol and Polysorbate 60);Polawax (Cetearyl Alcohol, PEG-150 Stearate, Polysorbate 60, and Steareth-20). A suitable proportion may be 75% water, 10% emulsifying wax, 5% other oil, and 10% sprayable antiviral composition as defined herein. Lotions and creams are typically oil or wax based, with the difference being that creams may be “heavier”; while gels are typically water based with a gelling agent (eg, xanthan gum, SDS, carrageenan, and so on). The exact composition of the base is not believed to be critical to the current invention, and the skilled person will be aware of suitable ingredients for cosmetic preparations for creams, lotions, or gels for topical application to the skin. Any dermatologically-acceptable carrier or base preparation may be used. Further, “neutral” cosmetic bases can be purchased ready-made from various suppliers.

In preferred embodiments, the sprayable antiviral composition includes a relatively high level of PMD (for example, 10%, 15%, 20%, 25%), to account for the dilution resulting from incorporation into a gel, cream, or lotion.

Thus, the present invention also provides a gel, cream, or lotion composition for topical application to the skin, the composition comprising an antiviral composition as described herein.

Also provided is a method of making a gel, cream or lotion composition for topical application to the skin, the method comprising providing a cosmetic gel, cream, or lotion base preparation, and combining the base preparation with an antiviral composition as described herein.

### EXAMPLES

The following examples are intended to illustrate aspects of the invention, and to describe how it may be practiced. They are not intended to limit the scope of the invention.

We aimed to identify ways to solubilise or emulsify PMD into a water-thin solution or emulsion that can be sprayed in a fog. Ideally the ingredients were to be readily available and the formula be suitable for cold-processing, thereby allowing production in many different environments and circumstances (for example, in temporary hospitals, or in remote or underserved areas of the world).

Our research made use of a commercially available PMD solution, from Chemical Process Laboratories, Silverton, South Africa which was specified as being 70-75% active by weight. On analysis, the test sample as received was 71% active. Our objective was to provide an emulsion/solution with 5% active content; hence the following examples indicate use of 7.15% PMD solution to give a final 5% active content. Alternative sources of PMD may of course be used, and in particular when scaling up for larger production runs it is possible to commercially obtain PMD Solution with a range of 55% to 75% and up to 95-97% pure PMD in solid form.

The reason for selecting 5% active is to ensure sufficient PMD is present in the applied final product, assuming some inevitable variation in surface skin moisture, inconsistency of application, and so on. However, PMD is believed to provide antiviral effects across a range of concentrations. The insect repellent effect is only seen at much higher concentrations, and hence most existing PMD-containing formulations are geared towards stability at these higher (up to 30%) concentrations.

Formula Development

Previous experience with Citriodiol in formulations for insect repellents was expected to be relevant in reaching the goal. However, the formulation process proved to be unexpectedly complex. One particular issue was that a blend of solubilisers that worked when added with Citriodiol at 30% did not work when in the same proportion with PMD Solution at 7.15%. After further consideration of potential formulations, some were prepared but overnight storage at 5C showed crystals developing in most of them; hence these were considered unsuitable.

After further development, four possible solutions were obtained, and were tested at 5C, ambient and 40C. After 5 days all were clear at ambient and 40C but three showed different levels of crystal development at 5C. Concurrent with trying to find clear solutions we also looked at various emulsifier possibilities and two were identified that appeared promising. These were also tested for 5 days at the 3 conditions. One emulsion showed signs of instability but the other one remained stable for the full 10 days test period.

Table 1 below shows the components of two test emulsions, and four test solutions, and the results after 10 days storage at the three temperature conditions. One emulsion (F1/7229B) appeared stable after 10 days at 40C and one solution (F1/7743C) showed no sign of crystals after 10 days at 5C. We determined that both propylene glycol (PG) and isopropanol (IPA) show solvent properties for PMD Solution.

TABLE 1
Tested Samples

| | Emulsions | | Solutions | | | |
|---|---|---|---|---|---|---|
| Ingredients | F1/7229B | F1/7229C | F1/7729A | F1/7743B | F1/7743C | F1/7743D |
| PMD | 7.15 | 7.15 | 7.15 | 7.15 | 7.15 | 7.15 |
| Solution | | | | | | |
| Propylene | 15.00 | 10.00 | 10.00 | 0 | 0 | 20.00 |
| Glycol | | | | | | |
| Easymuls | 2.50 | 0 | 0 | 0 | 0 | 0 |
| Plus* | | | | | | |
| Water | 75.35 | 80.35 | 42.85 | 47.85 | 61.35 | 56.55 |
| Lysofix | 0 | 2.5 | 0 | 0 | 0 | 0 |
| Liquid | | | | | | |
| Isopropanol | 0 | 0 | 30.00 | 0 | 15.00 | 0 |
| Polysorbate- | 0 | 0 | 10.00 | 0 | 0 | 0 |
| 60** | | | | | | |
| Polysorbate- | 0 | 0 | 0 | 0 | 16.50 | 16.30 |
| 80*** | | | | | | |
| Butylene | 0 | 0 | 0 | 30.00 | 0 | 0 |
| Glycol | | | | | | |
| Crodasol | 0 | 0 | 0 | 15.00 | 0 | 0 |
| WS | | | | | | |
| | 100% | 100% | 100% | 100% | 100% | 100% |

10-day Storage

| | | | | | | |
|---|---|---|---|---|---|---|
| 5 C. - | Clear | Crystals | V. slight | Cloudy | Clear | Slight |
| crystals? | | visible | crystals | | | crystals |
| Ambient | Stable | Stable | Clear | Clear | Clear | Clear |
| 40 C. | Stable | Unstable | Clear | Clear | Clear | Clear |

Following this investigation and previous screening processes Easymuls plus is the emulsifier of choice. It is Glyceryl Oleate Citrate from Evonik, described as a natural, anionic PEG-free 0/W emulsifier for low-viscous products. It shows excellent cold emulsifying capacities and can stabilise oil contents from 5% to 30% without significantly increasing the emulsion viscosity, making it very suitable for a spray application.

Of the many solubilisers tried Polysorbate-80 was the most effective but it was present at quite a high level. We next looked at reducing its level to 10% and adjusting levels of propylene glycol and isopropanol to find the optimum blend when used at 30% in a formulation—see Table 2.

TABLE 2
To find Optimum Blend of IPA/PG.

| | Blend 1 | Blend 2 | Blend 3 | Blend 4 |
|---|---|---|---|---|
| Ingredients | | | | |
| PMD Solution | 7.00 | 7.00 | 7.00 | 7.00 |
| Polysorbate-80 | 10.00 | 10.00 | 10.00 | 10.00 |
| IPA | 30.00 | 20.00 | 10.00 | 0.00 |
| PG | 0 | 10.00 | 20.00 | 30.00 |
| Water, deionised | 53.00 | 53.00 | 53.00 | 53.00 |
| Storage Results | | | | |
| 5 C. | Clear | Clear | Cloudy | Cloudy |
| Ambient | Clear | Clear | when | when |
| 40 C. | Clear | Clear | mixed | mixed |

We were left with one emulsion and three solutions that appeared to fulfil our requirements, they remained stable for a minimum of 10 days and were each sprayed through perfume atomisers without a problem.

We only had one emulsion under test and previous work showed the advantage of using a polar ester to improve stability and inhibit crystal formation. Further samples were prepared using three different esters; C12-15 Alkyl Benzoate, Decyl Oleate and Ethylhexyl Carbonate and these were put under test as before.

There are three solutions; the preference is for F1-7744 B using both PG and IPA. PG will slow evaporation rate and inhibit crystals forming in the spray nozzle after use.

TABLE 3

| | Emulsions | | | | Solutions | | |
|---|---|---|---|---|---|---|---|
| Ingredients | F1/7229B | F1/7764A | F1/7764B | F1/7764C | F1/7743C | F1/7744 A | F1/7744 B |
| PMD | 7.15 | 7.15 | 7.15 | 7.15 | 7.15 | 7.15 | 7.15 |
| Solution | | | | | | | |
| Propylene | 15.00 | 10.00 | 10.00 | 10.00 | 0 | 0 | 10.00 |
| Glycol | | | | | | | |
| Easymuls | 2.50 | 3.00 | 3.00 | 3.00 | 0 | 0 | 0 |
| Plus* | | | | | | | |
| C12-15 Alkyl | 0 | 5.00 | 0 | 0 | 0 | 0 | 0 |
| benzoate | | | | | | | |
| Decyl Oleate | 0 | 0 | 5.00 | 0 | 0 | 0 | 0 |
| Diethyl Hexyl | 0 | 0 | 0 | 5.00 | 0 | 0 | 0 |
| Carbonate | | | | | | | |
| Water | 75.35 | 74.85 | 74.85 | 74.85 | 61.35 | 52.85 | 52.85 |
| Isopropanol | 0 | 0 | 0 | 0 | 15.00 | 30.00 | 20.00 |
| Polysorbate- | 0 | 0 | 0 | 0 | 16.50 | 10.00 | 10.00 |
| 80 | | | | | | | |
| | 100% | 100% | 100% | 100% | 100% | 100% | 100% |

Experiments have also been carried out on preparing concentrates for emulsions. Examples of concentrates are shown in Table 4 below.

TABLE 4
Concentrates - to be diluted 40% concentrate/60% water.

| Ingredients | F1/7764A | F1/7764B |
|---|---|---|
| PMD Solution | 17.875 | 17.875 |
| Propylene Glycol | 37.5 | 37.5 |
| Easymuls Plus* | 7.125 | 7.125 |
| C12-15 Alkyl benzoate | 12.50 | 0 |
| Decyl Oleate | 0 | 12.50 |
| Water | 25.00 | 25.00 |
| | 100% | 100% |

Barrier Preparations

Having prepared various stable formulations comprising PMD, primarily with the aim of making a sprayable antiviral composition, we realised that the stable formulation could additionally be used as a base ingredient in potentially longer-lasting barrier preparations (for example, skin creams, lotions, or gels). These have the advantage of possibly improved compliance (as they can be directly applied to the skin rather than sprayed in a general environmental misting), longer lasting effects (given that the barrier preparation will remain on the skin rather than evaporating), and a wider range of applications. Further, this retains the advantages of the stable preparation, since this may be prepared at one location, and combined with a cream, lotion, or gel base preparation at another, to allow for suitable distribution networks.

Accordingly, we will test the emulsions, solutions, and concentrates described herein in combination with cosmetic bases for creams, lotions, and gels. Even where the initial stable preparation contains 5% PMD, this is believed to still be capable of providing antiviral effects even when diluted ten- or twenty-fold in a cream, gel, or lotion base.

One example cream formulation for topical application comprises

| ingredient | % weight |
|---|---|
| Xanthan gum | 0.3 |
| PMD 100% active | 5 |
| C12-15 Alkyl Benzoate | 5 |
| Propylene Glycol | 10 |
| Capric/Caprylic Triglyceride | 5 |
| Diisopropyl Adipate | 10 |
| OLIVEM 1000 (Cetearyl Olivate + | 4.5 |
| Sorbitan Olivate) | |
| OLIWAX LC (Cetyl Palmitate, | 4.5 |
| Sorbitan Palmitate, Sorbitan | |
| Olivate) | |
| Water | To 100% |

Virucidal Activity Tests

Compositions according to the invention were tested for residual virucidal activity against coronaviruses, using a test method based on ASTM E3058-16. A 5% PMD sprayable composition, also containing 10% propylene glycol, 20% isopropanol, and 10% polysorbate 80, and the remainder being water, was shown to provide a log reduction of 3.13 (>99.9%) against Human coronavirus HCoV-229E (ATCC VR-740) after 4 hours residual time from a 5 minute contact time on simulated skin testing substrate (IMS® VITRO-SKIN). Similarly, a 5% PMD cream formulation according to the invention provided a log 3 reduction after 4 hours residual time from 5 minute contact time.

The cream formulation was also tested for immediate virucidal activity against HCoV-229E on the simulated skin substrate, using a test method based on ASTM E1838-17. A 5 minute contact time resulted in a log 3.25 reduction.

This confirms the antiviral activity of the compositions of the invention. We note in passing that U.S. Pat. No. 7,872,051 referred to herein reported a maximum log reduction of 2.5 against Urbani SARS virus using PMD alone. This suggests that the compositions of the invention show a more effective virucidal activity than the active PMD alone; without wishing to be bound by theory, we hypothesise that one or more of the other ingredients in the compositions is able to have some effect on the viral lipid envelope, providing either an additive or a synergistic effect.

Further, bactericidal testing of the sprayable composition in accordance with EN 1276:2019 showed a reduction of greater than log 5 against E. coli with a contact time of 60 seconds, and lower but still significant reductions against E. hirae, S. aureus, and P. aeruginosa. This confirms the suitability of the composition to be used as a bactericidal formulation, as well as virucidal.

1. An antiviral composition, the composition comprising:
a) from 0.1% to 25% by weight of the total composition of p-Menthane-3,8-diol (PMD);
b) from 5% to 40% by weight of the total composition of one or more solvents selected from short chain (C1-05) primary and short chain (C3-05) secondary alcohols, and combinations thereof;
c) either i) from 0.5% to 10% by weight of the total composition of a glyceride-based emulsifier; or
ii) from 10% to 30% by weight of the total composition of a non-ionic ethoxylated sorbitan ester surfactant.

2. The composition of claim 1, further comprising:
d) from 2% to 10% by weight of a polar ester.

3. The composition of claim 1 or 2, further comprising:
e) from 0.1% to 3% of an emulsion stabiliser.

4. The composition of any preceding claim, consisting essentially of components a) to c) and water, and optionally components d) and/or e).

5. The composition of any preceding claim comprising 5% by weight of PMD.

6. The composition of any preceding claim wherein the solvent is selected from propan-2-ol and propane-1,2-diol.

7. The composition of any preceding claim wherein the solvent comprises from 5% to 20% by weight of the total weight of the composition.

8. The composition of any preceding claim wherein a mix of solvents is used, and the mix comprises 25-35% by weight of the total weight of the composition.

9. The composition of claim 8 wherein the composition comprises 25% propan-2-ol and 5% propane-1,2-diol, or wherein the composition comprises 20% propan-2-ol and 10% propane-1,2-diol.

10. The composition of any preceding claim wherein the solvent is or includes propane-1,2-diol, and component c) is not an emulsifier.

11. The composition of any of claims 1-9 wherein the glyceride-based emulsifier is present at 1.5-3.5% by weight.

12. The composition of any preceding claim wherein the non-ionic ethoxylated sorbitan ester surfactant is present at 10-20% by weight.

13. The composition of any preceding claim wherein the non-ionic ethoxylated sorbitan ester surfactant comprises polysorbate 80.

14. An antiviral composition, the composition comprising:
a) from 0.5% to 10%, preferably 3-7%, more preferably 5%, by weight of the total composition of p-Menthane-3,8-diol (PMD);
b) from 10% to 30%, preferably 15-20%, more preferably 20%, by weight of the total composition of propan-2-ol;
c) from 10% to 30%, preferably 10%, by weight of the total composition of polysorbate 80.

15. An antiviral composition, the composition comprising:
a) from 0.5% to 10%, preferably 3-7%, more preferably 5%, by weight of the total composition of p-Menthane-3,8-diol (PMD);
b) 20% by weight of the total composition of propan-2-ol;
c) from 10% to 30%, preferably 10%, by weight of the total composition of polysorbate 80; and
d) 10% by weight of the total composition of propane-1,2-diol.

16. An antiviral composition, the composition comprising:
a) 5% by weight of the total composition of p-Menthane-3,8-diol (PMD);
b) 15-30% by weight of the total composition of propan-2-ol;
c) from 10% to 20% by weight of the total composition of polysorbate 80; and
d) 0-10% by weight of the total composition of propane-1,2-diol; wherein, when propane-1,2-diol is present, the total combined amount of propane-1,2-diol and propan-2-01 is 30%.

17. An antiviral composition, the composition comprising:
a) 5% by weight of the total composition of p-Menthane-3,8-diol (PMD);
b) 10-15% by weight of the total composition of propane-1,2-diol;
c) 0-5% by weight of the total composition of a polar ester;
d) 1-5% by weight of the total composition of an emulsifier; and.
e) 0.1% to 3% by weight of the total composition of an emulsion stabiliser.

18. A spray bottle containing a composition as defined in any preceding claim.

19. A gel, cream, or lotion composition for topical application to the skin, the composition comprising an antiviral composition as defined in any of claims 1-17.

20. A method of making a gel, cream or lotion composition for topical application to the skin, the method comprising providing a cosmetic gel, cream, or lotion base preparation, and combining the base preparation with an antiviral composition as defined in any of claims 1-17.
